(19) United States
(12) Patent Application Publication
(10) Pub. No.: US 2017/0367681 A1
(43) Pub. Date: Dec. 28, 2017

(54) ULTRASONIC SENSING APPARATUS AND SENSING METHOD THEREOF

(71) Applicant: MiiCs & Partners (Shenzhen) Co., Ltd., Shenzhen (CN)

(72) Inventor: JUAN WANG, Shenzhen (CN)

(21) Appl. No.: 15/338,538

(22) Filed: Oct. 31, 2016

(30) Foreign Application Priority Data
Jun. 27, 2016 (CN) 201610476754.3

(51) Int. Cl. A61B 8/00

(52) U.S. Cl. CPC A61B 8/4494

(57) ABSTRACT

An ultrasonic sensing apparatus held next to a target can detect vital signs of the target. The ultrasonic sensing apparatus includes a first flexible circuit board, a transmitting layer, a readout layer, and a receiving layer. Emitting elements in the transmitting layer generate ultrasonic signals and the receiving layer receives reflected ultrasonic signals and converts the reflections into electrical signals. The emitting elements are staggered in relation to elements of the receiving layer for better resolution and accuracy. The readout layer reads the electric signals for calculating vital signs. The readout layer includes input lines and output lines and readout pixels are defined by the crossing points of input and output lines. Ultrasonic signals are generated during a first period, and the reflections are read in a second period next following. The readout layer completes one reading operation corresponding to one readout pixel during the second period.

### CROSS-REFERENCE TO RELATED APPLICATIONS

This application claims priority to Chinese Patent Application No. 201610476754.3 filed on Jun. 27, 2016, the contents of which are incorporated by reference herein.

### FIELD

The subject matter herein generally relates to an ultrasonic sensing apparatus and a sensing method related to the ultrasonic sensing apparatus.

### BACKGROUND

An ultrasonic sensing apparatus can sense a heart rate. The ultrasonic sensing apparatus can include a transmitting layer, a receiving layer, a readout layer, a first flexible printed circuit (FPC), a second FPC, and a third FPC. The transmitting layer generates ultrasonic signals output by the first FPC and the second FPC. The receiving layer receives ultrasonic signals reflected by an organism and converts the received ultrasonic signals into electrical signals. The readout layer analyzes the electrical signals to obtain vital signs of the organism. However, accuracy of the ultrasonic sensing apparatus would be improved by a better structure. Therefore, there is room for improvement in the art.

### BRIEF DESCRIPTION OF THE FIGURES

Implementations of the present technology will now be described, by way of example only, with reference to the attached figures.

FIG. 1 is an exploded view of a first exemplary embodiment of an ultrasonic sensing apparatus comprising a transmitting layer and a readout layer.

FIG. 2 is a plan view of the readout layer of FIG. 1.

FIG. 3 is a waveform of the transmitting layer and the readout layer of FIG. 1.

FIG. 4 is an exploded view of a second exemplary embodiment of an ultrasonic sensing apparatus.

FIG. 5 is a cross-sectional view of the ultrasonic sensing apparatus of FIG. 4, the ultrasonic sensing apparatus comprising a plurality of transmitting sources.

FIG. 6 is a cross-sectional view of a first exemplary embodiment of the transmitting sources of FIG. 5 with different amplitudes and a specified phase.

FIG. 7 is a cross-sectional view of a second exemplary embodiment of the transmitting sources of FIG. 5 with a specified amplitude and a specified phase.

FIG. 8 is an exploded view of a third exemplary embodiment of an ultrasonic sensing apparatus comprising a transmitting layer and a readout layer.

FIG. 9 is a cross-sectional view of the ultrasonic sensing apparatus of FIG. 8.

FIG. 10 is a waveform of the transmitting layer and the readout layer of FIG. 8.

FIG. 11 is a partially exploded view of a fourth exemplary embodiment of an ultrasonic sensing apparatus.

FIG. 12 is a partially exploded view of a fifth exemplary embodiment of an ultrasonic sensing apparatus.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The term “substantially” is defined to be essentially conforming to the particular dimension, shape, or other feature that the term modifies, such that the component need not be exact. For example, “substantially cylindrical” means that the object resembles a cylinder, but can have one or more deviations from a true cylinder. The term “comprising” means “including, but not necessarily limited to”; it specifically indicates open-ended inclusion or membership in a so-described combination, group, series, and the like.

The present disclosure is described in relation to an ultrasonic sensing apparatus.

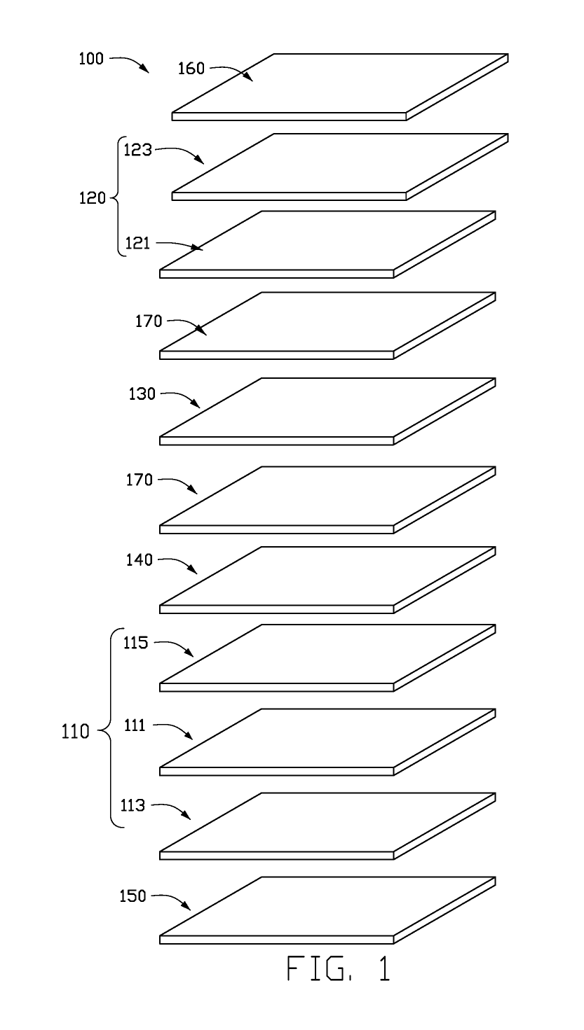

FIG. 1 illustrates a first exemplary embodiment of an ultrasonic sensing apparatus 100. The ultrasonic sensing apparatus 100 senses vital signs of humans, for example, a heart rate, a pulse, a blood pressure, and so on. The ultrasonic sensing apparatus 100 can be a tethered apparatus to paste onto skin, or can be embedded in a smart watch or a smart wrist strap.

The ultrasonic sensing apparatus 100 includes a transmitting layer 110, a receiving layer 120, a readout layer 130, a first flexible circuit board 140, a second flexible circuit board 150, a third flexible circuit board 160, and two adhesive layers 170. In other embodiments, the ultrasonic sensing apparatus 100 includes an adhesive layer for sticking the ultrasonic sensing apparatus 100 onto a target.

The transmitting layer 110 includes a transmitting element 111, a first conductive structure 113, and a second conductive structure 115. The transmitting element 111 is positioned between the first conductive structure 113 and the second conductive structure 115. The first conductive structure 113 is positioned between the transmitting element 111 and the second flexible circuit board 150. The second conductive structure 115 is positioned between the transmitting element 111 and the first flexible circuit board 140. The first conductive structure 113 and the second conductive structure 115 together drive the transmitting element 111 to generate ultrasonic signals. In at least one embodiment, the transmitting element 111 is made of piezoelectric material, for example, polyvinylidene fluoride (PVDF), BaiO₃, PbiO₃, Pb(Zri)O₃, plumbum scandium tantalite (PST), quartz, (Pb, Sm)iO₃, PMN(Pb(MgNb)O₃)—PT(PbiO₃), or PVDF-TrFE. In at least one embodiment, the first conductive structure 113 and the second conductive structure 115 are substantially flat. The first conductive structure 113 and the second conductive structure 115 can be formed on a surface of the transmitting element 111 by a vacuum sputtering manner, a painting manner, or a coating manner. In at least one embodiment, opposite surfaces of the transmitting element 111 can be conductively pasted to the first conductive structure 113 and the second conductive structure 115.

The receiving layer 120 includes a receiving element 121 and a third conductive structure 123. The receiving element 121 is positioned between the third conductive structure 123 and the two adhesive layers 170. The receiving element 121 receives the ultrasonic signals reflected by the target. The third conductive structure 123 is positioned between the receiving element 121 and the third flexible circuit board 160. The third conductive structure 123 converts the received ultrasonic signals into electric signals. In at least one embodiment, the receiving element 121 is also made of piezoelectric material, for example, the same material as that of the transmitting element 111. In at least one embodiment, the third conductive structure 123 is substantially flat. In other embodiments, the third conductive structure 123 can include a plurality of sensing electrodes separated from each other. Each of the sensing electrodes can be a rectangular shape, a wave shape, a serrated shape, or other shape. The third conductive structure 123 can be formed on a surface of the receiving element 121 by a vacuum sputtering manner, a painting manner, or a coating manner. In at least one embodiment, the receiving element 121 can be conductively pasted to the third conductive structure 123.

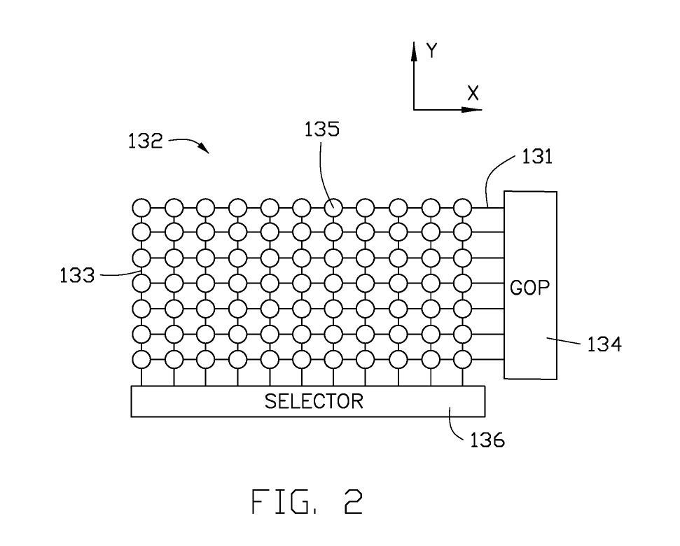

Referring to FIG. 2, the readout layer 130 can read electrical signals transmitted from the third conductive structure 123 and then transmit the electrical signals to a readout circuit (not shown) for calculating vital signs. The readout layer 130 is made of flexible material to adjust to the shape or surface of a target. The readout layer 130 includes a readout unit 132. The readout unit 132 includes a plurality of input lines 131 extending along a first direction X, a plurality of output lines 133 extending along a second direction Y, a gate driver on panel (GOP) 134, and a selector 136. The second direction Y is perpendicular to the first direction X. Readout pixels 135 are defined by the output lines 131 intersecting with the output lines 133. The input lines 131 are electrically connected to the GOP 134, and the output lines 133 are electrically connected to the selector 136. The GOP 134 selects the input lines 131 to turn on the readout pixels 135 for receiving electrical signals from the receiving layer 120. The selector 136 selects the output lines 133 to provide a visual display of the electrical signals. In at least one embodiment, the selector 136 is a multiplexer.

The first flexible circuit board 140 is electrically connected to the second conductive structure 115, and the second flexible circuit board 150 is electrically connected to the first conductive structure 113 and the second conductive structure 115. The first flexible circuit board 140 provides a first specified voltage to the second conductive structure 115, and the second flexible circuit board 150 provides a second specified voltage to the first conductive structure 113. The second specified voltage is different from the first specified voltage. The third flexible circuit 160 provides data to the third conductive structure 123. In at least one embodiment, the first flexible circuit board 140, the second flexible circuit board 150, and the third flexible circuit board 160 are insulated from each other. In other embodiments, the first flexible circuit board 140, the second flexible circuit board 150, and the third flexible circuit board 160 can be electrically connected.

One of the two adhesive layers 170 is positioned between the receiving element 121 and the readout layer 130 for pasting the receiving layer 120 on the readout layer 130. Another of the two adhesive layers 170 is positioned between second conductive structure 115 and the readout layer 130 for pasting first conductive layer 113 on the readout layer 130.

When the ultrasonic sensing apparatus 100 is in contact with a target, such as a wrist, a voltage difference between the first conductive structure 113 and the second conductive structure 115 causes the transmitting element 111 to vibrate to generate ultrasonic signals. The ultrasonic signals pass through the receiving layer 120 and the third flexible circuit board 160 to reach the target. The ultrasonic signals which reach the target are partially reflected by the target or by components within the target. The receiving layer 121 receives the reflected ultrasonic signals. The third conductive structure 123 converts the ultrasonic signals into electrical signals for transmission to the readout layer 130. The target can be in direct contact with the third flexible circuit board 160, or spaced apart from the third flexible circuit board 160 by a specified distance.

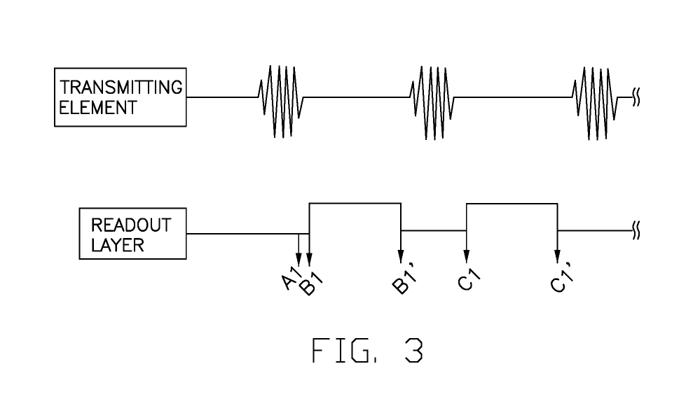

FIG. 3 illustrates a waveform of the ultrasonic sensing apparatus 100. The ultrasonic sensing apparatus 100 works between a first period and a second period. The transmitting layer 110 generates the ultrasonic signals in the first period, and stops generating the ultrasonic signals during the second period. The readout layer 130 reads the electronic signals on the output lines 133 in the second period, and does not read the electronic signals on the output lines 133 in the first period. During the second period, the selector 136 completes one read operation corresponding to one of the readout pixels 135. In detail, the GOP 134 sequentially turns on the readout pixel 135, and the selector 136 sequentially reads the electronic signals on the output lines 133. The selector 136 further calculates a difference between the received electronic signals and a specified value. Node A1 represents a starting time of generating the ultrasonic signals of the transmitting layer 110. Node B1 represents a starting time of turning on the readout pixels 135 connected to the first input line 131 for receiving the electronic signals after conversion from the reflected ultrasonic signals. Node B1′ represents an ending time of reading the electronic signals of the readout pixel 135 connected to the first output line 133. Node C1 represents a starting time of turning on the readout pixels 135 connected to the second input line 131 for receiving the electronic signals. Node C1′ represents an ending time of reading the electronic signals of the readout pixel 135 connected to the second output line 133. The time interval between the node B1 and the node C1 is more than 10 μs.

Based on the time sequence, a sensing depth of the ultrasonic sensing apparatus 100 is improved.

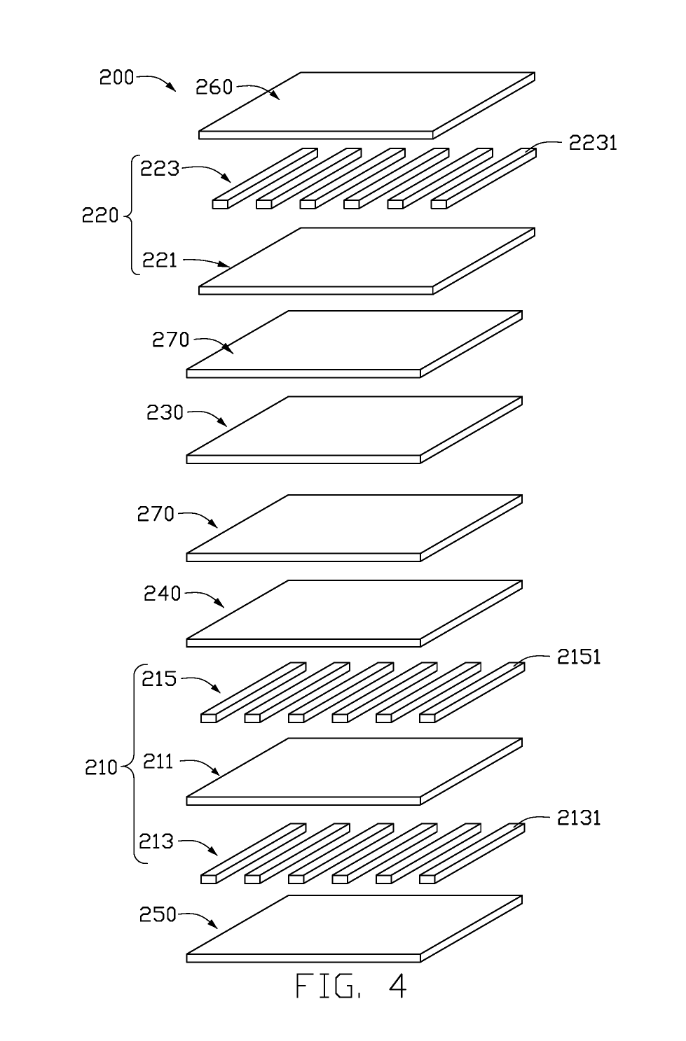

FIG. 4 illustrates a second exemplary embodiment of an ultrasonic sensing apparatus 200. The ultrasonic sensing apparatus 200 according to the second exemplary embodiment is substantially the same as the ultrasonic sensing apparatus 100. The ultrasonic sensing apparatus 200 includes a transmitting layer 210, a receiving layer 220, a readout layer 230, a first flexible circuit board 240, a second flexible circuit board 250, a third flexible circuit board 260, and two adhesive layers 270.

The differences between the ultrasonic sensing apparatus 200 and the ultrasonic sensing apparatus 100 are hereinafter described.

The transmitting layer 210 includes a transmitting element 211, a first conductive structure 213, and a second conductive structure 215. The transmitting element 211 is positioned between the first conductive structure 213 and the second conductive structure 215. The first conductive structure 213 is positioned between the transmitting element 211 and the second flexible circuit board 250. The second conductive structure 215 is positioned between the transmitting element 211 and the first flexible circuit board 240. The first conductive structure 213 and the second conductive structure 215 together drive the transmitting element 211 to generate ultrasonic signals.

The first conductive structure 213 includes a plurality of first sensing electrodes 2131 parallel with each other, the plurality extends along the second direction Y. The second conductive structure 215 includes a plurality of second sensing electrodes 2151 parallel with each other, this plurality extends along the second direction Y. Each of the first sensing electrodes 2131 faces and is aligned with one second sensing electrode 2151. The first sensing electrodes 2131 and the second sensing electrodes 2151 are substantially strip shaped. In at least one embodiment, the transmitting element 211 is made of the same piezoelectric material as hereinbefore described. In other embodiments, the first sensing electrodes 2131 and the second sensing electrodes 2151 can be a wave shape, or a serrated shape, or other shape. The first conductive structure 213 and the second conductive structure 215 can be formed on opposite surfaces of the transmitting element 211 by a vacuum sputtering manner, a painting manner, or a coating manner. In at least one embodiment, opposite surfaces of the transmitting element 211 can be conductively pasted to the first conductive structure 213 and the second conductive structure 215.

The receiving layer 220 includes a receiving element 221 and a third conductive structure 223. The receiving element 221 is positioned between the third conductive structure 223 and one of the two adhesive layers 270. The receiving element 221 receives ultrasonic signals reflected by the target. The third conductive structure 223 is positioned between the receiving element 221 and the third flexible circuit board 260. The third conductive structure 223 converts the received ultrasonic signals into electric signals. The third conductive structure 223 includes a plurality of third sensing electrodes 2231 parallel with each other, which extends along the second direction Y. Each of the third sensing electrodes 2231 faces and is aligned with one first sensing electrode 2131. In at least one embodiment, the third sensing electrode 2231 is substantially strip shaped. In at least one embodiment, the receiving element 221 is made of the same piezoelectric material as hereinbefore described. In other embodiments, the third sensing electrodes 2231 are staggered in relation to the first sensing electrodes 2131, the electrodes 2231 can be a wave shape, or a serrated shaped, or other shape. The third conductive structure 223 can be formed on a surface of the receiving element 221 by a vacuum sputtering manner, a painting manner, or a coating manner. In at least one embodiment, the receiving element 221 can be conductively pasted to the third conductive structure 223.

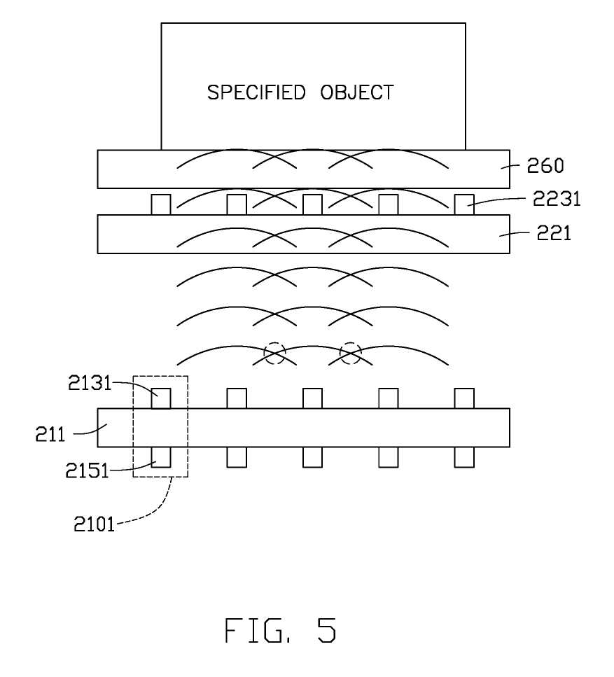

FIG. 5 illustrates the first exemplary embodiment of the ultrasonic sensing apparatus 200 in cross section. Each first sensing electrode 2131 and a corresponding second sensing electrode 2151 together form a transmitting source 2101, thereby forming a plurality of transmitting sources 2101 as the first conductive structure 213 and the second conductive structure 215. Ultrasonic signals from all emitter components of all the transmitting sources 2101 can be generated simultaneously.

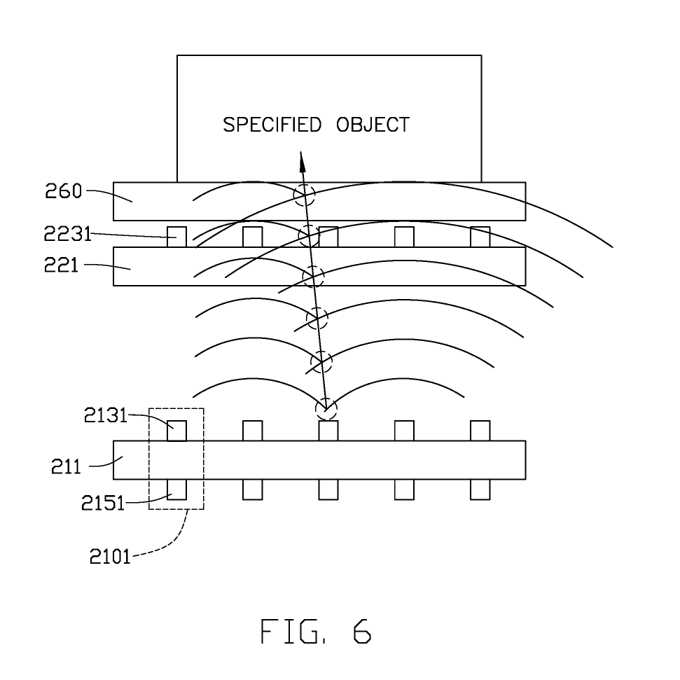

FIG. 6 illustrates the transmitting sources 2101 outputting with different specified amplitudes and a specified phase. The voltages applied on the first sensing electrodes 2131 and on the second sensing electrodes 2151 are different, thus the ultrasonic signals generated by different components of a transmitting source 2101 are in different amplitudes, but in a same specified phase. An intensity of the ultrasonic signals generated by two adjacent components in a transmitting source 2101 is enhanced at crossed points arranged in a line inclined with the transmitting element 211.

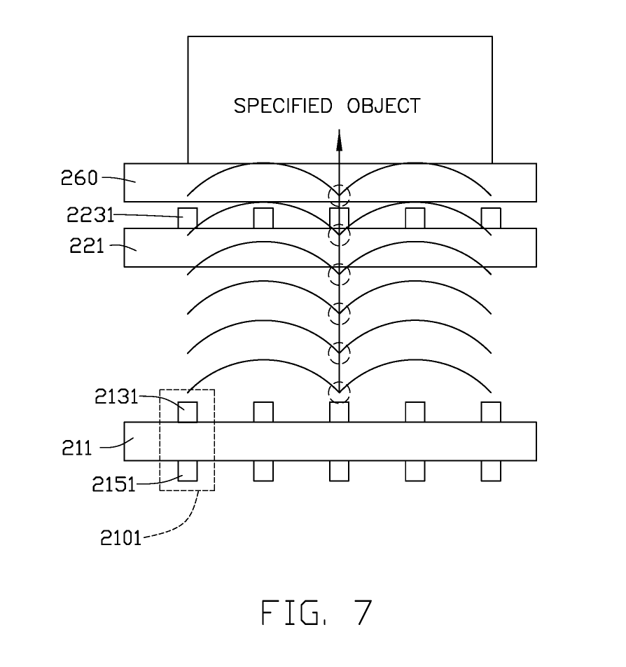

FIG. 7 illustrates the transmitting sources 2101 with a specified amplitude and a specified phase. The voltage applied on the first sensing electrodes 2131 and the second sensing electrodes 2151 are the same, thus all of the ultrasonic signals generated by the transmitting sources 2101 are in a specified amplitude and a specified phase. An intensity of the ultrasonic signals generated by two adjacent components of within a transmitting source 2101 is enhanced at crossed points arranged in a line perpendicular to the transmitting element 211.

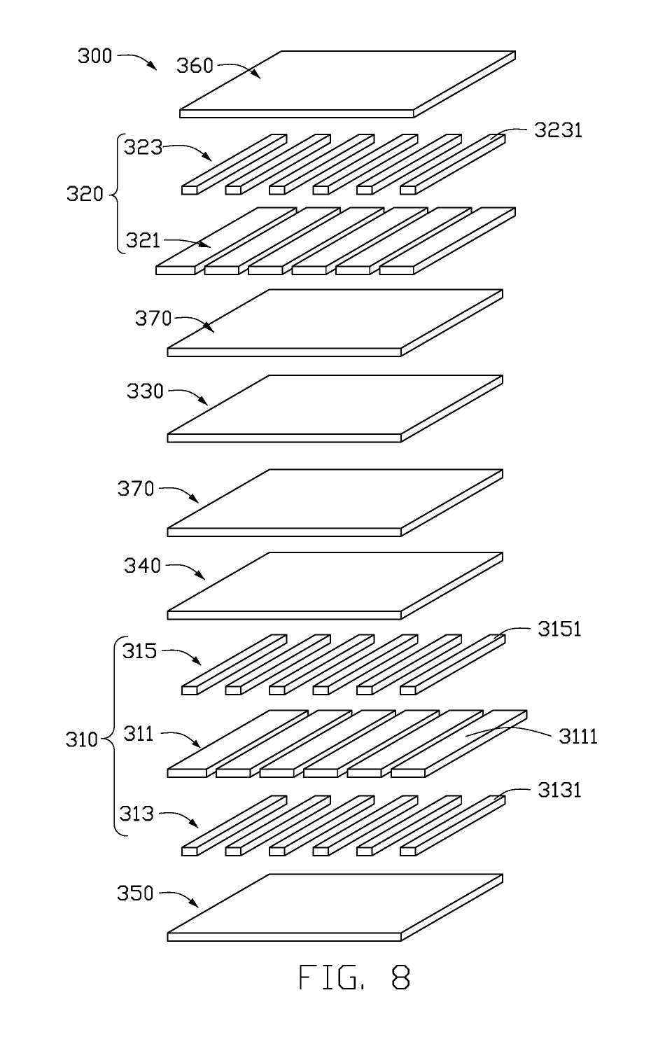

FIG. 8 illustrates a third exemplary embodiment of the ultrasonic sensing apparatus 300. The ultrasonic sensing apparatus 300 according to the second exemplary embodiment is substantially the same as the ultrasonic sensing apparatus 100. The ultrasonic sensing apparatus 300 includes a transmitting layer 310, a receiving layer 320, a readout layer 330, a first flexible circuit board 340, a second flexible circuit board 350, a third flexible circuit board 360, and two adhesive layers 370.

The differences between the ultrasonic sensing apparatus 300 and the ultrasonic sensing apparatus 100 are hereinafter described.

The transmitting layer 310 includes a transmitting element 311, a first conductive structure 313, and a second conductive structure 315. The transmitting element 311 is positioned between the first conductive structure 313 and the second conductive structure 315. The transmitting element 311 includes a plurality of transmitting units 3111 parallel with each other, the plurality extending along the second direction Y. The transmitting unit 3111 is substantially strip shaped. The first conductive structure 313 is positioned between the transmitting element 311 and the second flexible circuit board 350. The second conductive structure 315 is positioned between the transmitting element 311 and the first flexible circuit board 340. The first conductive structure 313 and the second conductive structure 315 together drive the transmitting element 311 to generate ultrasonic signals. The first conductive structure 313 includes a plurality of first sensing electrodes 3131 parallel with each other, the plurality extending along the second direction Y, and the second conductive structure 315 includes a plurality of second sensing electrodes 3151 parallel with each other, this plurality extending along the second direction Y. Each of the first sensing electrodes 3131 faces and is aligned with one second sensing electrode 3151. Each of the transmitting units 3111 is positioned between one of the first sensing electrodes 3131 and the second sensing electrode 3151 which it faces. The first sensing electrodes 3131 and the second sensing electrodes 3151 are substantially strip shaped. In at least one embodiment, the transmitting element 111 is made of the same piezoelectric material as hereinbefore described. In other embodiments, the first sensing electrodes 3131 and the second sensing electrodes 3151 can be a wave shape, or a serrated shape, or other shape. The first conductive structure 313 and the second conductive structure 315 can be formed on opposite surfaces of the transmitting element 311 by a vacuum sputtering manner, a painting manner, or a coating manner. In at least one embodiment, opposite surfaces of the transmitting element 311 can be conductively pasted to the first conductive structure 313 and the second conductive structure 315.

The receiving layer 320 includes a receiving element 321 and a third conductive structure 323. The receiving element 321 is positioned between the third conductive structure 323 and one of the two adhesive layers 370. The receiving element 321 receives the ultrasonic signals reflected by the target. The receiving element 321 includes a plurality of receiving units 3211 parallel with each other, the plurality extending along the second direction Y. Each of the receiving unit 3211 is substantially strip shaped. Each of the receiving units 3211 faces and is aligned with one first sensing electrode 3131. The third conductive structure 323 is positioned between the receiving element 321 and the third flexible circuit board 360. The third conductive structure 323 converts the received ultrasonic signals into electric signals. The third conductive structure 323 includes a plurality of third sensing electrodes 3231 parallel with each other, the plurality extending along the second direction Y. Each of the third sensing electrodes 3231 faces and is aligned with one first sensing electrode 3131. In at least one embodiment, the third sensing electrode 3231 is substantially strip shaped. In at least one embodiment, the receiving element 321 is made of the same piezoelectric material as hereinbefore described. In other embodiments, the third sensing electrodes 3231 are staggered in relation to the first sensing electrodes 3131, and can be a wave shape, or a serrated shaped, or other shape. The third conductive structure 323 can be formed on a surface of the receiving element 321 by a vacuum sputtering manner, a painting manner, or a coated manner. In at least one embodiment, the receiving element 321 can be conductively pasted to the third conductive structure 323.

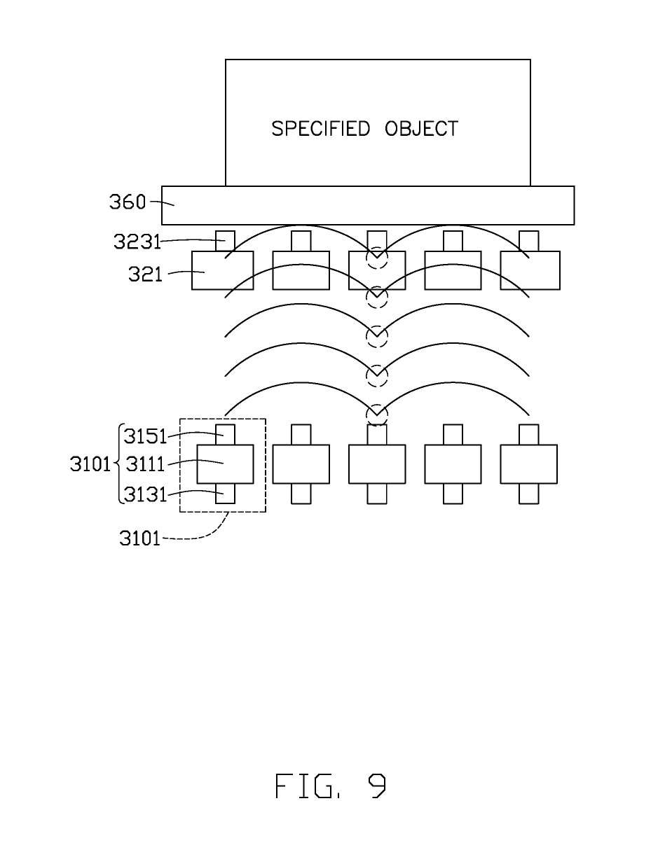

FIG. 9 illustrates a plan view of the ultrasonic sensing apparatus 300. The first sensing electrode 3131, the transmitting unit 3111, and the second sensing electrode 3151 together form a transmitting source 3101. All emitting components of the transmitting source 3101 simultaneously generate the ultrasonic signals. The ultrasonic signals generated by two adjacent transmitting sources 3101 will be enhanced at crossing points.

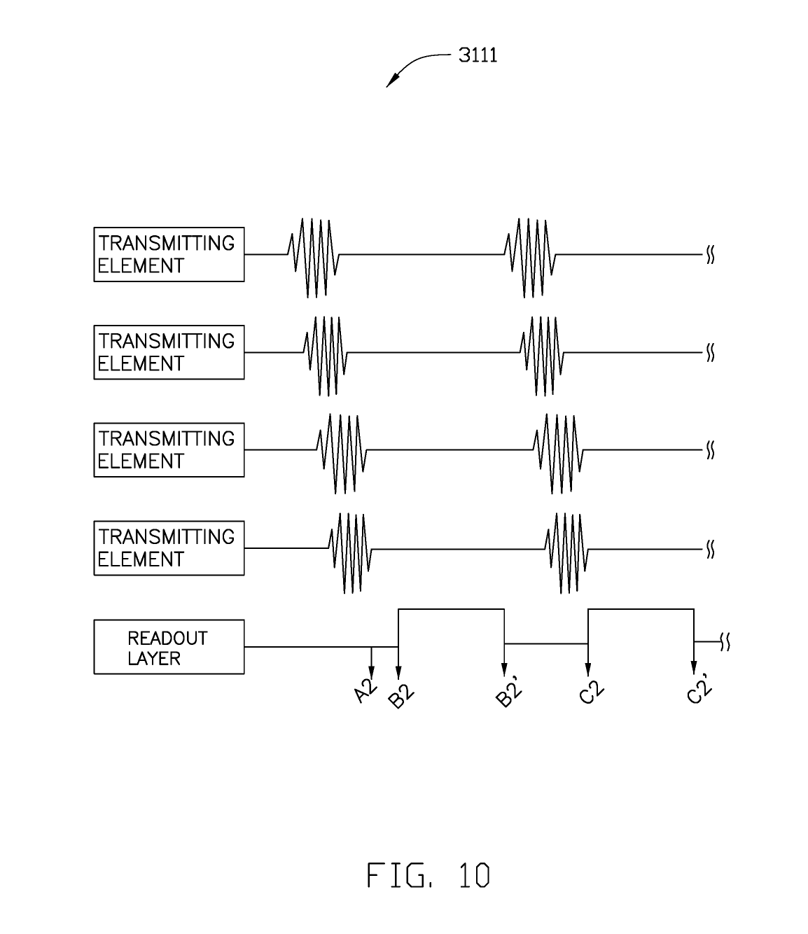

FIG. 10 shows a time sequence of the ultrasonic sensing apparatus 300. The transmitting units 3101 output the ultrasonic signals in series during the first period. The first periods of the transmitting units 3101 are overlapped with each other. The readout layer 330 reads the electronic signals in the second period, and does not read the electronic signals in the first period. During the second period, the selector 336 completes one reading operation corresponding to one of the readout pixel 335.

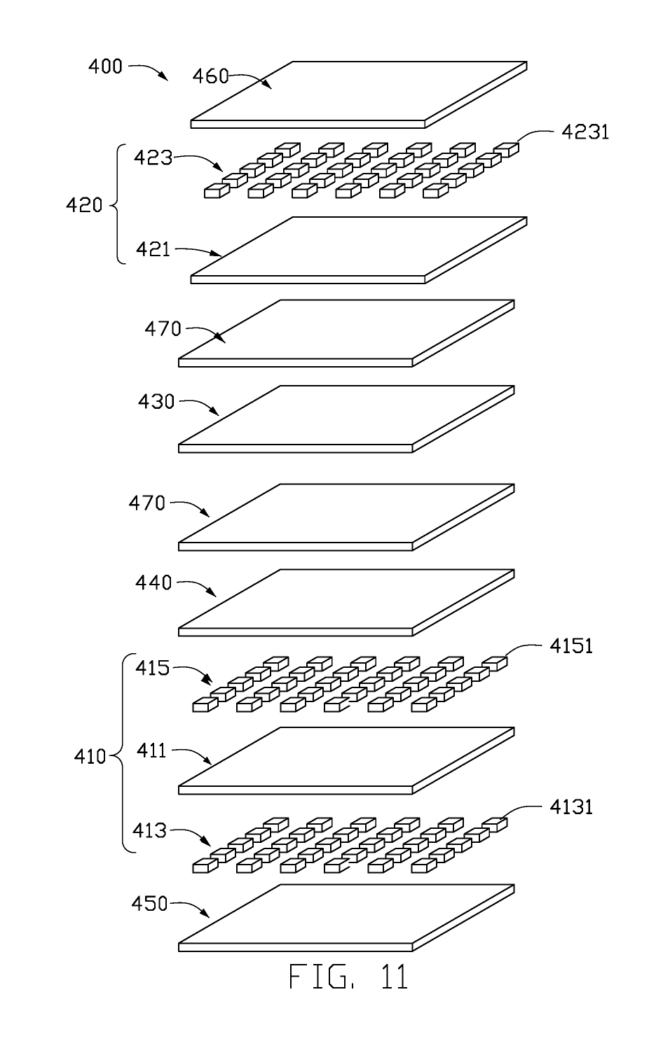

FIG. 11 a fourth exemplary embodiment of the ultrasonic sensing apparatus 400. The ultrasonic sensing apparatus 400 according to the fourth exemplary embodiment is substantially the same as the ultrasonic sensing apparatus 100. The ultrasonic sensing apparatus 400 includes a transmitting layer 410, a receiving layer 420, a readout layer 430, a first flexible circuit board 440, a second flexible circuit board 450, a third flexible circuit board 460, and two adhesive layers 470.

The differences between the ultrasonic sensing apparatus 400 and the ultrasonic sensing apparatus 100 are hereinafter described.

The transmitting layer 410 includes a transmitting element 411, a first conductive structure 413, and a second conductive structure 415. The transmitting element 411 is positioned between the first conductive structure 413 and the second conductive structure 415. The first conductive structure 413 is positioned between the transmitting element 411 and the second flexible circuit board 450. The second conductive structure 415 is positioned between the transmitting element 411 and the first flexible circuit board 440. The first conductive structure 413 and the second conductive structure 415 together drive the transmitting element 411 to generate ultrasonic signals. The first conductive structure 413 includes a plurality of first sensing electrodes 4131 in a matrix, and the second conductive structure 415 includes a plurality of second sensing electrodes 4151 in a matrix. Each of the first sensing electrodes 4131 faces one second sensing electrode 4151. The first sensing electrodes 4131 and the second sensing electrodes 4151 are substantially square shaped. In at least one embodiment, the transmitting element 111 is made of the same piezoelectric material as hereinbefore described. In other embodiments, the first sensing electrodes 4131 and the second sensing electrodes 4151 can be a wave shape, or a serrated shape, or other shape. The first conductive structure 413 and the second conductive structure 415 can be formed on a surface of the transmitting element 411 by a vacuum sputtering manner, a painting manner, or a coating manner. In at least one embodiment, opposite surfaces of the transmitting element 411 can be conductively pasted to the first conductive structure 413 and the second conductive structure 415.

The receiving layer 420 includes a receiving element 421 and a third conductive structure 423. The receiving element 421 is positioned between the third conductive structure 423 and the two adhesive layers 470. The receiving element 421 receives the ultrasonic signals reflected by the target. The third conductive structure 423 is positioned between the receiving element 421 and the third flexible circuit board 460. The third conductive structure 423 converts the received ultrasonic signals into electric signals. The third conductive structure 423 includes a plurality of third sensing electrodes 4231 in a matrix. Each of the third sensing electrodes 4231 faces one first sensing electrode 4131. In at least one embodiment, the third sensing electrode 4231 is substantially square shaped. In at least one embodiment, the receiving element 421 is made of the same piezoelectric material as hereinbefore described. In other embodiments, the third sensing electrodes 4231 are staggered in relation to the first sensing electrodes 4131, and can be a wave shape, or a serrated shape, or other shape. The third conductive structure 423 can be formed on a surface of the receiving element 421 by a vacuum sputtering manner, a painting manner, or a coating manner. In at least one embodiment, the receiving element 421 can be conductively pasted to the third conductive structure 423.

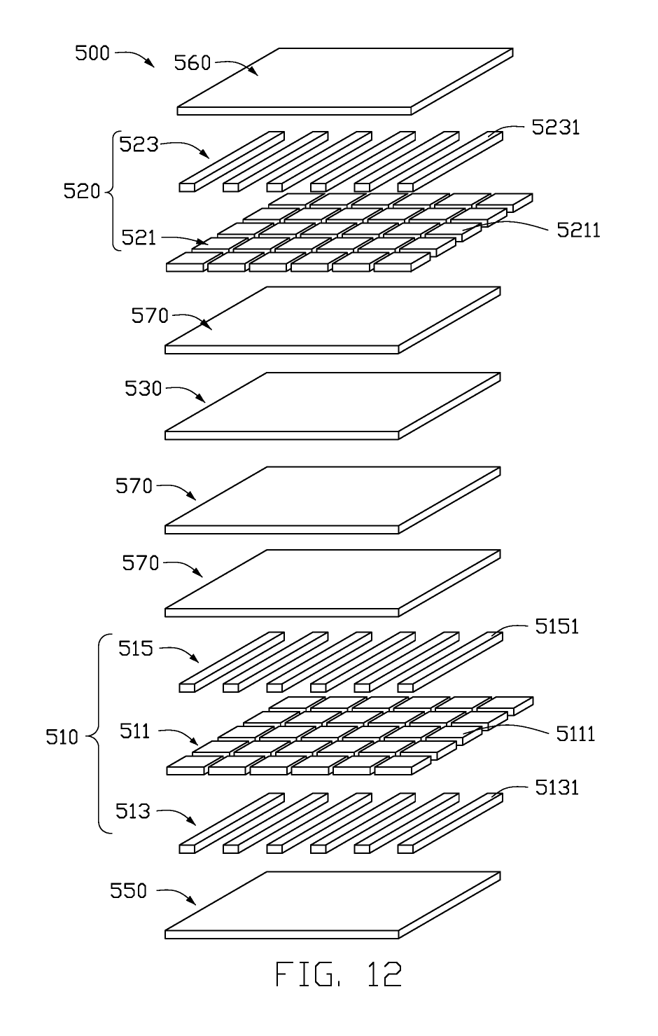

FIG. 12 a fifth exemplary embodiment of the ultrasonic sensing apparatus 500. The ultrasonic sensing apparatus 500 according to the fifth exemplary embodiment is approximately the same as the ultrasonic sensing apparatus 100. The ultrasonic sensing apparatus 500 includes a readout layer 530, a first flexible circuit board 540, a second flexible circuit board 550, a third flexible circuit board 560, and two adhesive layers 570.

The differences between the ultrasonic sensing apparatus 500 and the ultrasonic sensing apparatus 100 are hereinafter described.

The transmitting layer 510 includes a transmitting element 511, a first conductive structure 513, and a second conductive structure 515. The transmitting element 511 is positioned between the first conductive structure 513 and the second conductive structure 515. The transmitting element 511 includes a plurality of transmitting units 5111 in a matrix. The transmitting unit 5111 is substantially square shaped. The first conductive structure 513 is positioned between the transmitting element 511 and the second flexible circuit board 550. The second conductive structure 515 is positioned between the transmitting element 511 and the first flexible circuit board 540. The first conductive structure 513 and the second conductive structure 515 together drive the transmitting element 511 to generate ultrasonic signals. The first conductive structure 513 includes a plurality of first sensing electrodes 5131 parallel with each other, the plurality extending along the second direction Y, and the second conductive structure 515 includes a plurality of second sensing electrodes 5151 parallel with each other, this plurality extending along the second direction Y. Each of the first sensing electrodes 5131 faces and is aligned with one second sensing electrode 5151. The first sensing electrodes 5131 and the second sensing electrodes 5151 are substantially strip shaped. In at least one embodiment, the transmitting element 111 is made of the same piezoelectric material as hereinbefore described. In other embodiments, the first sensing electrodes 5131 and the second sensing electrodes 5151 can be a wave shape, or a serrated shape, or other shape. The first conductive structure 513 and the second conductive structure 515 can be formed on a surface of the transmitting element 511 by a vacuum sputtering manner, a painting manner, or a coating manner. In at least one embodiment, opposite surfaces of the transmitting element 511 can be conductively pasted to the first conductive structure 513 and the second conductive structure 515.

The receiving layer 520 includes a receiving element 521 and a third conductive structure 523. The receiving element 521 is positioned between the third conductive structure 523 and the two adhesive layers 570. The receiving element 521 receives the ultrasonic signals reflected by the target. The receiving element 521 includes a plurality of receiving units 5211 in a matrix. The receiving unit 5211 is substantially square shaped. Each of the receiving units 5211 faces one transmitting unit 5111. The third conductive structure 523 is positioned between the receiving element 521 and the third flexible circuit board 560. The third conductive structure 523 converts the received ultrasonic signals into electric signals. The third conductive structure 523 includes a plurality of third sensing electrodes 5231 parallel with each other, the plurality extending along the second direction Y. Each of the third sensing electrodes 5231 faces and is aligned with one first sensing electrode 5131. In at least one embodiment, the third sensing electrode 5231 is substantially strip shaped. In at least one embodiment, the receiving element 521 is made of the same piezoelectric material as hereinbefore described. In other embodiments, the third sensing electrodes 5231 are staggered in relation to the first sensing electrodes 5131, and can be a wave shape, or a serrated shape, or other shape. The third conductive structure 523 can be formed on a surface of the receiving element 521 by a vacuum sputtering manner, a painting manner, or a coating manner. In at least one embodiment, the receiving element 521 can be conductively pasted to the third conductive structure 523.

While various exemplary and preferred embodiments have been described, the disclosure is not limited thereto. On the contrary, various modifications and similar arrangements (as would be apparent to those skilled in the art) are intended to also be covered. Therefore, the scope of the appended claims should be accorded the broadest interpretation so as to encompass all such modifications and similar arrangements.

What is claimed is:

1. An ultrasonic sensing apparatus pasted on a target for detecting vital signs of the target, the ultrasonic apparatus comprising:
a transmitting layer configured to generate ultrasonic signals;
a receiving layer disposed on a surface of the transmitting layer, the receiving layer configured to receive ultrasonic signals reflected by the target and convert the ultrasonic signals into electrical signals; and
a readout layer positioned between the transmitting layer and the receiving layer, the readout layer configured to read the electric signals for calculating vital signs, wherein the readout layer comprises:
a plurality of input lines extending along a first direction; and
a plurality of output lines extending along a second direction;
wherein readout pixels are defined by the output lines intersecting with the output lines; the transmitting layer generates the ultrasonic signals during a first period, and stops generating the ultrasonic signals during a second period; the readout layer reads the electronic signals in the second period, and stops reading the electronic signals in the first period; wherein the readout layer completes one reading operation corresponding to one of the readout pixels during the second period.

2. The ultrasonic sensing apparatus of claim 1, wherein the transmitting layer comprises a first conductive structure, a second conductive structure, and a transmitting element; the first conductive structure comprises a plurality of first sensing electrodes, and the second conductive structure comprises a plurality of second sensing electrodes; each of the first sensing electrodes faces one of the second sensing electrodes.

3. The ultrasonic sensing apparatus of claim 2, wherein each of the first sensing electrodes cooperates with the corresponding second sensing electrode and the transmitting element to form a transmitting source.

4. The ultrasonic sensing apparatus of claim 3, wherein the ultrasonic signals generated by the transmitting sources are in different amplitudes, but are in a same specified phase; the ultrasonic signals generated by two adjacent transmitting sources are enhanced at crossed points arranged in a line inclined with the transmitting element.

5. The ultrasonic sensing apparatus of claim 3, wherein the ultrasonic signals generated by the transmitting sources are in different amplitudes and are in a same specified phase; the ultrasonic signals generated by two adjacent transmitting sources are enhanced at crossed points arranged in a line perpendicular to the transmitting element.

6. The ultrasonic sensing apparatus of claim 2, wherein the transmitting element comprises a plurality of transmitting units; each of the transmitting units is positioned between one of the first sensing electrodes and the corresponding second sensing electrode; wherein the first sensing electrodes cooperate with the corresponding second sensing electrode and the corresponding transmitting element to form a transmitting source.

7. The ultrasonic sensing apparatus of claim 6, wherein the receiving layer comprises a receiving element and a third conductive structure; wherein the third conductive structure comprises a plurality of third sensing electrodes parallel with each other and extending along the second direction; wherein each of the third sensing electrodes faces one of the first sensing electrodes.

8. The ultrasonic sensing apparatus of claim 7, wherein the receiving element comprises a plurality of receiving units; each of the receiving units faces one of the transmitting units.

9. The ultrasonic sensing apparatus of claim 8, wherein the transmitting units are arranged in a matrix, and the receiving units are arranged in a matrix.

10. The ultrasonic sensing apparatus of claim 6, wherein the first sensing electrodes are parallel with each other and extend along the second direction; the second sensing electrodes are parallel with each other and extend along the second direction; the third sensing electrodes are parallel with each other and extend along the second direction.

11. The ultrasonic sensing apparatus of claim 6, wherein the first sensing electrodes are arranged in a matrix; the second sensing electrodes are arranged in a matrix; the third sensing electrodes are arranged in a matrix.

12. The ultrasonic sensing apparatus of claim 1, wherein the readout layer further comprises a gate driver on panel (GOP) electrically connected to the input lines and a selector electrically connected to the output lines; the GOP sequentially selects one of the input lines for simultaneously turning on the readout pixels connected to the selected input line; the selector selectively reads electrical signals on the readout pixels connected to the selected output lines.

13. An ultrasonic sensing apparatus pasted on a target for detecting vital signs of the target, the ultrasonic apparatus comprising:
a transmitting layer configured to generate ultrasonic signals;
a receiving layer disposed on a surface of the transmitting layer, the receiving layer configured to receive ultrasonic signals reflected by the target, and convert the ultrasonic signals into electrical signals; and
a readout layer positioned between the transmitting layer and the receiving layer, the readout layer configured to read the electric signals for calculating vital signs;
wherein the transmitting layer comprises a first conductive structure, a second conductive structure, and a transmitting element; the first conductive structure comprises a plurality of first sensing electrodes, and the second conductive structure comprises a plurality of second sensing electrodes; wherein each of the first sensing electrodes faces one of the second sensing electrodes; each of the first sensing electrodes cooperates with the corresponding second sensing electrode and the transmitting element to form a transmitting source; the ultrasonic signals generated by two adjacent transmitting sources are enhanced at crossed points.

14. The ultrasonic sensing apparatus of claim 13, wherein the crossed points are arranged in a line perpendicular to the transmitting element.

15. The ultrasonic sensing apparatus of claim 13, wherein the crossed points are arranged in a line inclined with the transmitting element.

16. The ultrasonic sensing apparatus of claim 13, wherein transmitting element comprises a plurality of transmitting units; each of the transmitting units is positioned between one of the first sensing electrodes and the corresponding second sensing electrode; the first sensing electrodes cooperate with the faced second sensing electrode and the corresponding transmitting element to form a transmitting source.

17. The ultrasonic sensing apparatus of claim 16, wherein the transmitting units are arranged in a matrix, and the receiving units are arranged in a matrix.

18. The ultrasonic sensing apparatus of claim 16, wherein the receiving layer comprises a receiving element and a third conductive structure; wherein the third conductive structure comprises a plurality of third sensing electrodes parallel with each other and extending along the second direction; wherein each of the third sensing electrodes faces one of the first sensing electrodes.

19. The ultrasonic sensing apparatus of claim 18, wherein the receiving element comprises a plurality of receiving units; each of the receiving units faces one of the transmitting units.

20. The ultrasonic sensing apparatus of claim 13, wherein the first sensing electrodes are parallel with each other and extend along the second direction; the second sensing electrodes are parallel with each other and extend along the second direction; the third sensing electrodes are parallel with each other and extend along the second direction.

21. The ultrasonic sensing apparatus of claim 13, wherein the first sensing electrodes are arranged in a matrix; the second sensing electrodes are arranged in a matrix; the third sensing electrodes are arranged in a matrix.

22. The ultrasonic sensing apparatus of claim 13, wherein the readout layer comprises a plurality of input lines extending along a first direction and a plurality of output lines which extend along a second direction; the readout pixels are defined by the output lines intersecting with the output lines; the transmitting layer generates the ultrasonic signals during a first period, and the readout layer reads the electronic signals in a second period; the readout layer completes one reading operation corresponding to one of the readout pixels during the second period.

23. The ultrasonic sensing apparatus of claim 22, wherein the readout layer further comprises a gate driver on panel (GOP) electrically connected to the input lines and a selector electrically connected to the output lines; the GOP sequentially selects one of the input lines for simultaneously turning on the readout pixels connected to the selected input line; the selector selectively reads electrical signals on the readout pixels connected to the selected output lines.
